(19) United States
(12) Patent Application Publication
(10) Pub. No.: US 2011/0087042 A1
(43) Pub. Date: Apr. 14, 2011

(54) CRYSTALLINE OXYBUTYNIN AND PROCESS FOR PREPARING THE SAME

(75) Inventors: Chandra Purna Ray, Jinnaram Mandal (IN); Madhuresh Sethi, Jinnaram Mandal (IN); Sanjay Mahajan, Jinnaram Mandal (IN); Om Dutt Tyagi, Jinnaram Mandal (IN)

(73) Assignee: MATRIX LABORATORIES LIMITED, Secunderabad, Andhra Pradesh (IN)

(21) Appl. No.: 12/866,002

(22) PCT Filed: Feb. 4, 2009
(86) PCT No.: PCT/IN2009/000074

§ 371 Date: Dec. 14, 2010

(30) Foreign Application Priority Data
Feb. 4, 2008 (IN) 297/CHE/2008

(51) Int. Cl. C07C 69/73

(52) U.S. Cl. 560/58

(57) ABSTRACT

The present invention relates to a crystalline oxybutynin base and process for preparing the same. Further, this invention discloses a process for preparing an acid addition salt of oxybutynin employing the crystalline oxybutynin base.

### FIELD OF THE INVENTION

This invention, relates to a crystalline oxybutynin base and process for preparing the same.

### BACKGROUND OF THE INVENTION

Oxybutynin and its derivatives are applicable as a bronchodilator or a remedy for pollakisuria. Also, oxybutynin exerts a direct antispasmodic effect on various forms of smooth muscle, mainly by inhibiting the action of acetylcholine on smooth muscle as an anti-cholinergic drug and the like. Oxybutynin is marketed in hydrochloride form. Oxybutynin known as [α-cyclohexyl-hydroxy-benzeneaceticacid-4-(diethyl amino)-2-butynyl ester] is given below:

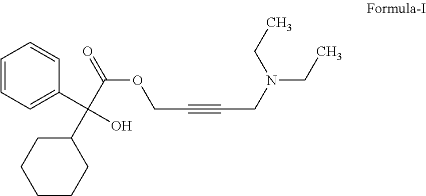

The U.S. Pat. No. 3,176,019 ('019) discloses about 4-amino-2-butynol esters and their derivatives, particularly about oxybutynin hydrochloride. It also reveals about the synthesis of oxybutynin, wherein, the methyl phenyl cyclohexyl glycolate is reacted with 4-diethylamino-2-butynylacetate in presence of base to yield oxybutynin followed by further workup. Further, it is treated with 2N HCl solution to form hydrochloride salt. It is recrystallised by employing ethyl acetate or water to obtain pure oxybutynin hydrochloride. Further, the US patent '019 unveils about the reaction of propargyl-2-cyclohexyl-2-hydroxy-2-phenyl acetate, p-formaldehyde and diethyl amine in dry dioxane to obtain crude product of oxybutynin. The dry hydrogen chloride gas is passed through the ether solution of oxybutynin to yield the oxybutynin chloride as precipitate.

According to the prior art process oxybutynin is obtained as oil, which contains lot of impurities, therefore, it needs to purify high vacuum distillation. Also, the resultant oxybutynin base is having a low melting point, which may decompose during high vacuum distillation. Further, the existence of any polymorphism in oxybutynin is not disclosed in prior arts. In light of the foregoing, a need exists in the art for inventing a new form and the process thereof.

### OBJECTS AND SUMMARY OF THE INVENTION

It is a principal object of the present invention is to provide a novel crystalline oxybutynin base in a solid state having improved quality.

Another object of the present invention is to provide a process for the preparation of novel crystalline oxybutynin base as a solid state.

Further, object of the present invention is to provide a process for preparing an acid addition salt of oxybutynin employing crystalline oxybutynin base

In accordance with one preferred embodiment of the present invention, there is provided a crystalline oxybutynin base characterized by using different analytical tools including X-ray powder diffraction pattern, Thermo Gravimetric Analysis (TGA), and Differential Scanning calorimetry (DSC).

In accordance with another preferred embodiment of the present invention there is provided a process for the preparation of crystalline oxybutynin base, wherein said process comprises of taking oxybutynin acid addition salt in a solvent, liberating the acid counter part from the acid addition salt of oxybutynin by the adjusting the pH employing a base to enable the isolation of the crystalline oxybutynin as its free base.

In accordance with one other preferred embodiment of the present invention, there is provided a process for preparing a crystalline oxybutynin base, wherein said process comprises of dissolving oxybutynin acid addition salt in a solvent, liberating the acid counter part from the acid addition salt of oxybutynin by the adjusting the pH employing a base, extracting the base with organic solvent, concentrating the resultant to obtain residue, treating the residue with non-polar solvent at low temperature and isolating crystalline oxybutynin.

In accordance with still another preferred embodiment of the present invention there is provided a process for the preparation of crystalline oxybutynin free base, wherein the process comprises of condensing the methyl phenyl cyclohexylglycolate and 4-diethylamino-2-butynyl-acetate and isolating the crystalline oxybutynin base as a solid state directly from the reaction mass, without preparing acid addition salt of oxybutynin.

In accordance with yet another preferred embodiment of the present invention there is provided a process for preparation of oxybutynin acid addition salt by reacting crystalline oxybutynin base obtained according to the present invention with acid to give pharmaceutically acceptable acid addition salt of oxybutynin.

### BRIEF DESCRIPTION OF THE DRAWINGS

Further objects of the present invention together with additional features contributing thereto and advantages accruing there from will be apparent from the following description of preferred embodiments of the invention which are shown in the accompanying drawing figures, wherein:

FIG. 1 is the X-ray powder diffraction pattern of crystalline oxybutynin free base.

FIG. 2 is the DSC of crystalline oxybutynin free base.

FIG. 3 is the TGA of crystalline oxybutynin free base.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention describes the crystalline oxybutynin free base, process for the preparation of the same. The said crystalline form of oxybutynin is characterized by their physical properties, spectral data which includes X-ray powder diffraction pattern, thermo gravimetric analysis (TGA), Differential Scanning Calorimetry (DSC) and IR absorption spectrum (IR).

### Powder X-ray Diffraction (PXRD)

The said polymorph of the present invention is characterized by their X-ray powder diffraction pattern. Thus, the X-ray diffraction patterns of said polymorph of the invention were measured on PANalytical, X'Pert PRO powder diffractometer equipped with goniometer of θ/θ configuration and X'Celerator detector. The Cu-anode X-ray tube was operated at 40 KV and 30 mA. The experiments were conducted over the 2θ range of 2.0°-5.0°, 0.030° step size and 50 seconds step time.

### Differential Scanning Calorimetry (DSC)

The DSC experiments were carried out on Mettler Toledo 822 Star and TA Q1000 of TA instruments. The experiments were performed at a heating rate of 10.0° C./min over a temperature range of 30° C.-300° C. purging with nitrogen at a flow rate of 150 ml/min and 50 ml/min. Standard aluminum crucibles covered by lids with three pin holes were used.

### DSC Glass Transition

The glass transition temperature (Tg) of the crystalline oxybutynin was measured on TA Q 1000 of TA instruments with modulated DSC software. The experiments were performed at a heating rate of 3.0° C./min up to a final temperature of 250° C. with modulation amplitude ±1.0° C., modulation period 80 sec and nitrogen purging at a flow rate of 50 ml/min. Standard aluminum crucibles covered by lids with five pin holes were used.

### Thermo Gravimetric Analysis (TGA)

TGA was recorded using the instrument Mettler Toledo TGA/SDTA 851^{e} and TA Q 5000 of TA instruments. The experiments were performed at a heating rate of 10.0° C./min over a temperature range of 30-300° C. purging with nitrogen at a flow rate of 20 ml/min and 25 ml/min.

According to the present invention, the crystalline oxybutynin is characterized by an X-ray powder diffraction pattern having peak at about 8.87±0.2, 10.76±0.2, 11.60±0.2, 14.22±0.2, 15.37±0.2, 15.90±0.2, 17.31±0.2, 17.65±0.2, 18.07±0.2, 19.09±0.2, 19.98±0.2, 20.58±0.2, 22.47±0.2, 22.74±0.2, 24.03±0.2, 24.30±0.2, 24.65±0.2, 25.15±0.2, 26.17±0.2, 26.61±0.2, 26.88±0.2, 28.23±0.2θ.

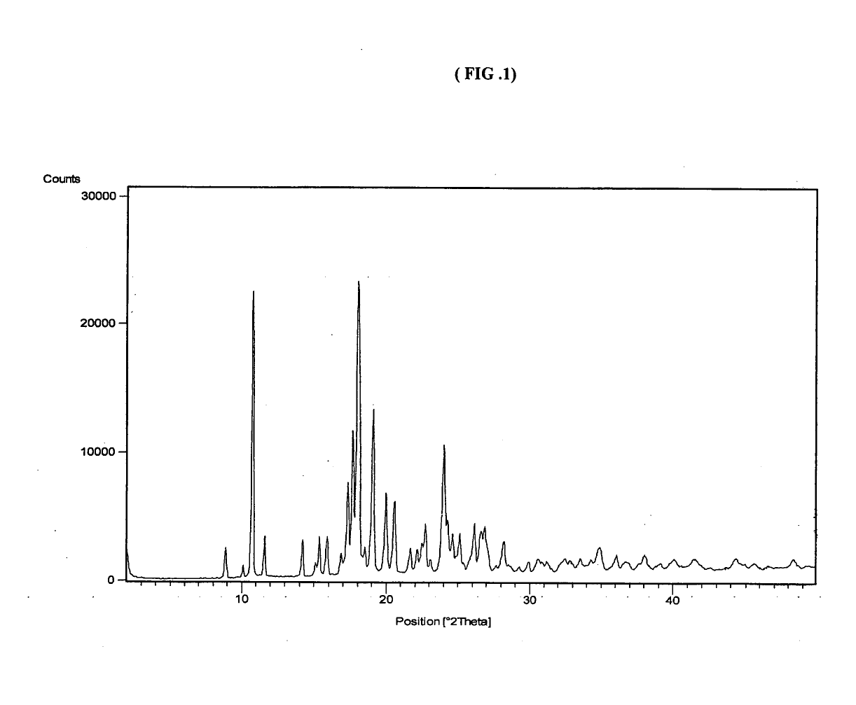

According to the present invention, the crystalline oxybutynin base is characterized by x-ray powder diffraction pattern shown in FIG. 1 having most discerning peaks at about 10.76±0.2, 18.079±0.2, 19.09±0.2, 24.03±0.2θ.

According to the present invention, the crystalline oxybutynin shows DSC peak as a sharp endotherm at 57.88° C. and TGA peak showing a weight loss of 0.1720%

According to the present invention, the process is provided for preparing crystalline oxybutynin base, wherein acid addition salt is dissolved in a solvent then pH is adjusted to above 8.0 with base, resulting solution is extracted with an organic solvent, which is concentrated under reduced pressure to get the residue, it is treated with non-polar solvent at low temperature and separated solid is cooled, filtered and washed with chilled pentane to give pure oxybutynin, wherein the solvent to dissolve the acid addition salt is selected from water or non-polar solvent selected from toluene, heptane, hexane, pentane or mixtures thereof, preferably water. The organic solvent to extract the said resulting solution is selected from heptane, hexane, pentane, toluene, ethyl acetate, dichloromethane, dichloroethane or chloroform preferably heptanes, wherein the preferable non-polar solvent for the isolation is selected from hexane, heptane, pentane or mixtures thereof, preferably pentane.

The adjustable pH according to the process disclosed herein preferably between about 8.0 to about 11.0. The base is used for the pH adjustment is selected from sodium bicarbonate, potassium bicarbonate, sodium carbonate, potassium carbonate, sodium hydroxide, potassium hydroxide, preferably sodium hydroxide.

According to the present invention, oxybutynin base is prepared directly from the reaction mass, comprising condensing methyl phenylcyclohexylglycolate and 4-diethylamino-2-butynyl acetate and isolating the crystalline oxybutynin base directly from the resultant reaction mass without preparing the oxybutynin acid addition salts.

Furthermore, the present invention involves usage of crystalline oxybutynin base obtained according to present invention for the preparation of pharmaceutically acceptable acid addition salts of oxybutynin by conventional method.

According to the present invention, the oxybutynin free base is isolate in solid state by treating with non-polar solvent at lower temperature in order to avoid the

According to the present invention, oxybutynin base is isolated as a solid by treating with non-polar solvent at low temperature. The isolated solid is separated by filtration to remove the impurities in mother liquor. The isolated solid is having the improved purity with subject to any further purification steps.

The following non-limiting examples illustrate specific embodiments of the present invention. They are, not intended to be limiting the scope of present invention in any way.

### Example-1

### Preparation of 4-diethylamino-2-butyne-ol

A mixture of para formaldehyde (105.0 g), N,N-diethyl amine (300 g) and copper(II) acetate (7.5 g) in 1,4 dioxane (900 ml) was heated to 60-65° C. After 1.5 h, 2-propyne-1-ol (150 g, 2.7 moles) was added and the mixture was heated at 90-95° C. after 2 hrs; excess solvent, 1,4 dioxane, evaporated at reduced pressure to afford 315 g (84%) of the product as an oil.

### Example-2

### Preparation of diethylamino-2-butynylacetate

A mixture of 4-diethylamino-2-butyne-1-ol (300 g), acetic acid (600 ml); acetic anhydride (300 ml) and con.sulphuric acid (15 ml) was heated to 65-70° C. After 2 hrs. of maintenance excess solvent mixture was evaporated at reduced pressure. The residue was cooled and poured in a mixture of dichloromethane (1800 ml) and DM water (3000 ml). The reaction mass was saturated with sodium bicarbonate (300 g) solid slowly controlling effervescences. The organic layer was separated and washed with 2% sodium bicarbonate and 1% EDTA solution to afford 318 g (81%) of product as oil.

### Example-3

### Preparation of 4-diethylamino-2-butynyl phenyl cyclohexyl glycolate hydrochloride (Oxybutynin Hydrochloride)

A mixture of 150 g of methyl phenyl cyclohexyl glycolate, 133 g of 4-diethylamino-2-butynyl acetate was dissolved in 1.8 hr of n-heptane. The solution was added with 1.2 g of sodium methoxide. The solution was heated with stirring to a temperature of 95-100° C. and distillate was collected. After 30 min of maintenance at 95-100° C., the solution was cooled to 65-70° C. under nitrogen. The solution was added with 3.24 g of sodium methoxide. The solution was heated with stirring to a temperature of 95-100° C. and distillate was collected. After 1 hr. maintenance at 95-100° C., reaction mass cooled to room temperature, washed with water. n-Heptane layer was separated and added 300 ml of 2N Hydrochloric acid to give oxybutynin hydrochloride. The crude was recrystallised from ethyl acetate.

### Example-4

### Preparation of Oxybutynin Base

A mixture of 150 g of methyl phenyl cyclohexyl glycolate, 133 g of 4-diethylamino-2-butynyl acetate was dissolved in 1.8 hr of n-heptane. The solution was added with 1.2 g of sodium methoxide. The solution was heated with stirring to a temperature of 95-100° C. and distillate was collected. After 30 min of maintenance at 95-100° C., the solution was cooled to 65-70° C. under nitrogen. The solution was added with 3.24 g of sodium methoxide. The solution was heated with stirring to a temperature of 95-100° C. and distillate was collected. After 1 hr. maintenance at 95-100° C., reaction mass cooled to room temperature, washed with water. n-Heptane layer was separated, concentrated under reduced pressure to give residue. n-Pentane (250 ml) was added to the residue and stirred under nitrogen atmosphere at 25-30° C. The solid product was filtered and washed with chilled n-pentane. Wet cake was dried at 40-42° C.

Dry weight=160.0 g

### Example-5

### Preparation of Oxybutynin (Base)

Oxybutynin chloride (100 gm) was treated with DM water (500 ml) at 25-30° C. and heated to 40-45° C. to observe clear solution. n-Heptane (500 ml) was added to the solution and adjusted the pH of the mass to 10.0-11.0 using 5% sodium hydroxide solution at 20-25° C. Layers obtained were separated and aqueous layer was extracted with heptane. Organic layers were combined and concentrated under vacuum at 40-45° C. to give residue. n-Pentane (250 ml) was added to the residue and stirred under nitrogen atmosphere at 25-30° C. The solid product was filtered and washed with chilled n-pentane. Wet cake was dried at 40-42° C.

Dry weight=85.0 gm

While this invention has been described in detail with reference to certain preferred embodiments, it should be appreciated that the present invention is not limited to those precise embodiments rather, in view of the present disclosure, which describes the current best mode for practicing the invention, many modifications and variations, would present themselves to those skilled in the art without departing from the scope and spirit of this invention.

1. A crystalline oxybutynin free base.

2. The crystalline oxybutynin free base according to claim 1, wherein the crystalline oxybutynin is characterized by an X-ray powder diffraction pattern having peaks at about 8.87±0.2, 10.76±0.2, 11.60±0.2, 14.22±0.2, 15.37±0.2, 15.90±0.2, 17.31±0.2, 17.65±0.2, 18.07±0.2, 19.09±0.2, 19.98±0.2, 20.58±0.2, 22.47±0.2, 22.74±0.2, 24.03±0.2, 24.30±0.2, 24.65±0.2, 25.15±0.2, 26.17±0.2, 26.61±0.2, 26.88±0.2, 28.23±0.2θ.

3. The crystalline oxybutynin according to claim 1, wherein the crystalline oxybutynin is characterized by an X-ray powder diffraction pattern having peak at about 10.76±0.2, 18.079±0.2, 19.09±0.2, 24.03±0.2θ.

4. The crystalline oxybutynin according to claim 1, wherein the crystalline oxybutynin is having a substantially similar X-ray powder diffraction pattern as shown in FIG. 1.

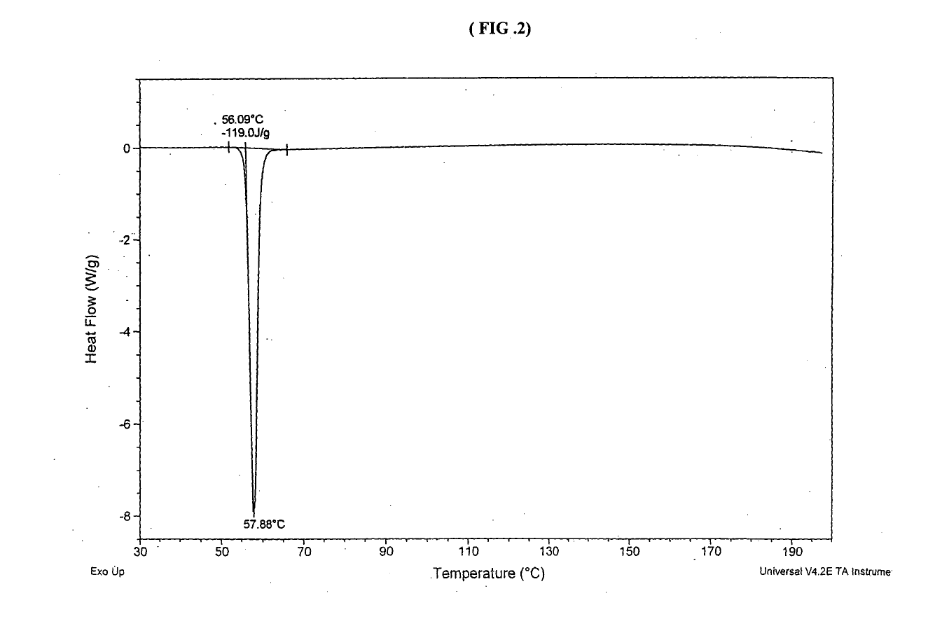

5. The crystalline oxybutynin according to claim 1, wherein the crystalline oxybutynin is characterized by a differential scanning calorimetry (DSC) as shown in FIG. 2.

6. The crystalline oxybutynin according to claim 5, wherein the crystalline oxybutynin is characterized by differential scanning calorimetry (DSC) is having a peak at about 57.88° C.

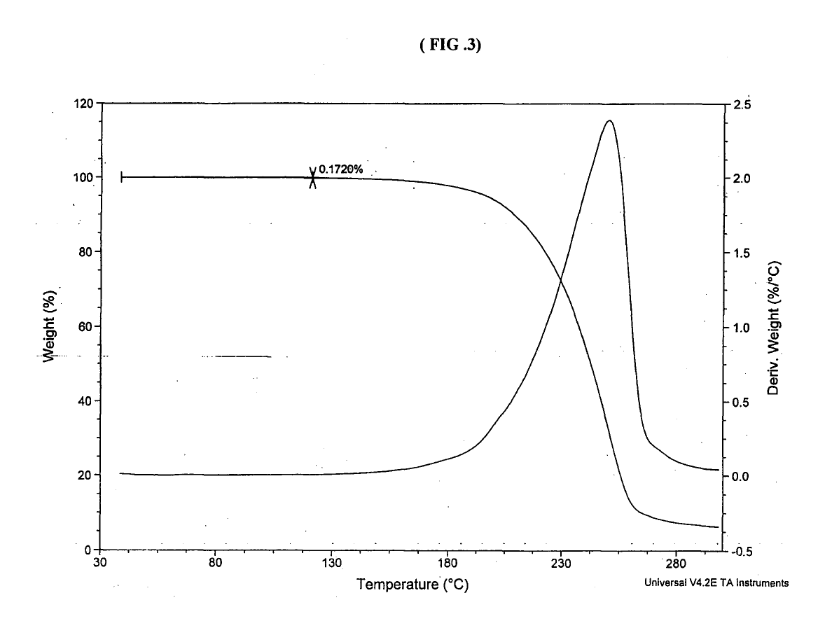

7. The crystalline oxybutynin according to claim 1, wherein the crystalline oxybutynin is characterized by a thermal gravimetric analysis (TGA) as shown in FIG. 3.

8. The crystalline oxybutynin according to claim 7, wherein the crystalline oxybutynin is characterized by thermal gravimetric analysis (TGA) having a weight loss of 0.172%.

9. A process for the preparation of crystalline oxybutynin comprising the steps of:
i) taking oxybutynin acid addition salt in a suitable solvent,
ii) liberating the acid from its acid addition salt of oxybutynin by adjusting the pH employing a base; and
iii) isolating the crystalline oxybutynin as its free base

10. The process according to claim 9, wherein the acid addition salt is selected from the group comprising hydrochloride, hydrobromide or sulfate.

11. The process according to claim 9, wherein the solvent is selected from water and non-polar organic solvents selected from hexane, heptane or in combination thereof.

12. The process according to claim 9, wherein the pH is adjusted to above 8.0.

13. The process according to claim 9, wherein the base is selected from the group comprising sodium bicarbonate, potassium bicarbonate, sodium carbonate, potassium carbonate, sodium hydroxide or potassium hydroxide most preferably, selected from sodium hydroxide.

14. A process for preparation of crystalline oxybutynin which comprising the steps of:
i) taking the oxybutynin acid addition salt in a solvent;
ii) liberating the acid from its acid addition salt of oxybutynin by adjusting the pH employing a base;
iii) extracting the resultant oxybutynin free base employing an organic solvent;
iv) concentrating the resultant extractions obtained in the above step (iii) to yield the crude residue of oxybutynin free base;
v) treating the resultant residue of oxybutynin free base employing a non-polar solvent; and
vi) isolating the pure crystalline oxybutynin as its free base.

15. The process according to claim 14, wherein the solvent is selected from n-heptane or water.

16. The process according to claim 14, wherein the pH is adjusted to above 8.0.

17. The process according to claim 14, wherein the organic solvent employed for the extraction is selected from dichloromethane, dichloroethane, chloroform, toluene, ethyl acetate, pentane, hexane, heptane or in combination thereof, preferably n-heptane.

18. The process according to claim 14, wherein the non-polar solvent employed for the isolation of the crystalline oxybutynin is selected from toluene, pentane, hexane, heptanes or in combination thereof, preferably n-pentane.

19. A process for preparation of crystalline oxybutynin base which comprises:
(i) condensing methyl phenylcyclohexylglycolate and 4-diethylamino-2-butynyl acetate; and
(ii) isolating the crystalline oxybutynin base from the resultant reaction mass directly.

20. A process for the preparation of oxybutynin acid addition salt, wherein the process comprises reacting the crystalline oxybutynin base obtained according to claim 19 with acid to give pharmaceutically acceptable acid addition salt of oxybutynin.

21. A process for the preparation of oxybutynin acid addition salt, wherein the process comprises reacting the crystalline oxybutynin base of claim 1 with acid to give pharmaceutically acceptable acid addition salt of oxybutynin.

22. A process for the preparation of oxybutynin acid addition salt, wherein the process comprises reacting the crystalline oxybutynin base obtained according to claim 9 with acid to give pharmaceutically acceptable acid addition salt of oxybutynin.

22. A process for the preparation of oxybutynin acid addition salt, wherein the process comprises reacting the crystalline oxybutynin base obtained according to claim 14 with acid to give pharmaceutically acceptable acid addition salt of oxybutynin.
